(19) United States
(12) Plant Patent Application Publication
(10) Pub. No.: US 2020/0029482 P1
(43) Pub. Date: Jan. 23, 2020

(54) Nectarine tree named 'SWEET REI'

(50) ;

(71) Applicant: Chukichi Takahashi, Date-shi (JP)

(72) Inventor: Chukichi Takahashi, Date-shi (JP)

(21) Appl. No.: 16/501,998

(22) Filed: Jul. 18, 2019

(30) Foreign Application Priority Data
Jul. 23, 2018 (JP) 33273

(51) Int. Cl. A01H 6/74

(52) U.S. Cl. PLT/188

(57) ABSTRACT

A new and distinct variety of white nectarine (Prunus persica (L.) Batsch var. nucipersica (Suckow) C. K. Schneid.) denominated ‘SWEET REI’, characterized by being late-maturing, having high Brix and relatively low acidity white flesh, excellent eating quality fruit, being easy to eat because of its freestone fruit, having an attractive dark-red fruit skin color, and no deterioration of eating quality of fruit even after storage for two weeks in a refrigerator.

Botanical classification: Prunus persica (L.) Batsch var. nucipersica (Suckow) C. K. Schneid.

Variety denomination: ‘SWEET REI’.

### CROSS-REFERENCE TO RELATED APPLICATION

This application claims priority of Japanese Plant Variety Registration Application No. 33273 filed on Jul. 23, 2018, which is incorporated by reference herein as if set forth in its entirety.

### BACKGROUND OF THE VARIETY

The present invention relates to a new and distinct variety of white nectarine (Prunus persica (L.) Batsch var. nucipersica (Suckow) C. K. Schneid.), which has been given the variety denomination ‘SWEET REI’. This novel variety was found by natural hybridization of the seed parent variety ‘SWEET VENUS’ (Japanese Plant Variety Registration No. 12301, registered on Nov. 8, 2004), in Tsukidate-cho, Date-city, Fukushima-prefecture, Japan.

This new white nectarine produces fruits, which are late-maturing, having high brix and relatively low acidity white flesh as well as attractive dark-red skin color, excellent eating quality, and are easy to eat because of its freestone flesh.

### ORIGIN OF THE VARIETY

As stated above, the new variety of white nectarine ‘SWEET REI’ was obtained from natural hybridization of ‘SWEET VENUS’ (Japanese Plant Variety Registration No. 12301, registered on Nov. 8, 2004), the seed parent of the present variety, in Tsukidate-cho, Date-city, Fukushima-prefecture, Japan, where it was tested.

In 2011, ‘SWEET VENUS’ was open-pollinated in the inventor's orchard in Tsukidate-cho, Date-city, Fukushima-prefecture, Japan, and 10 of its natural hybrid seeds were seeded and the resultant seedlings were grown at the orchard.

Five (5) germinated seedlings of the above-obtained seedlings were further grown, and the first fruits were observed in 2013. Among the 5 trees from the germinated seedlings, the inventor selected one tree as a mother tree because of its characteristics as having high brix (sweet) and relatively low acidity, excellent eating quality, and easy to eat fruits. In 2014, the second generation of the mother tree was grown by asexual reproduction by grafting onto a wild white-flowered peach tree rootstock (non-patented, being indigenous on a mountain in Tsukidate-cho, Date-city, Fukushima-prefecture, Japan), and the observation of the characteristics of the second generation tree was started. The asexually propagated tree remained true to the original tree (i.e., mother tree), and all characteristics of the tree and fruits of the mother tree were transmitted. In 2017, this new variety was denominated by the inventor as ‘SWEET REI’.

The above-identified mother tree and the second generation tree of ‘SWEET REI’ have been maintained in Tsukidate-cho, Date-city, Fukushima-prefecture, Japan.

### SUMMARY OF THE VARIETY

The new white nectarine variety ‘SWEET REI’ exhibits outstanding and distinguishing characteristics when grown under normal nectarine cultivation practices, including:
(1) high brix (Brix 15 to 17 degree), relatively low acidity, and white flesh fruit;
(2) excellent eating quality fruit;
(3) being easy to eat because of its freestone fruit;
(4) attractive dark-red fruit skin color; and
(5) no deterioration of eating quality of fruit even after storage for two weeks in a refrigerator.

The fruit of ‘SWEET REI’ is suitable for fruit gift, such as fruits gift box or fruits basket, since it has attractive dark-red skin color as well as excellent eating quality, and is easy to eat because of its freestone flesh.

In addition, since the fruit of ‘SWEET REI’ has high brix and relatively low acidity, it can also be used for preparation of processed fruits, such as fruit cake, fruit jelly, canned fruits, fruit juice and fruit jam.

For reliable description, the characteristics of ‘SWEET REI’ are described as compared to those of the yellow nectarine variety ‘SWEET VENUS’ (the seed parent of the present variety, Prunus persica (L.) Batsch var. nucipersica (Suckow) C. K. Schneid.) and the representative variety of white peach, ‘Kawanakajimahakuto’ (Prunus persica (L.) Batsch), in the attached Table of Characteristics.

As stated herein below and described in the attached Table of Characteristics, ‘SWEET REI’ has apparently different characteristics from those of the variety ‘SWEET VENUS’ and the variety ‘Kawanakajimahakuto’.

‘SWEET REI’ clearly differs from its seed parent variety ‘SWEET VENUS’, which has yellow, relatively firm and clingstone fruits, in that ‘SWEET REI’ has dark-red fruit skin color, white fruit flesh, and relatively soft, freestone and easy to eat fruit. Moreover, since ‘SWEET REI’ needs not being grown under plastic cover, its cultivation is easier than that of ‘SWEET VENUS’, which needs to be plastic covered.

In addition, ‘SWEET REI’ clearly differs from the representative variety of white peach ‘Kawanakajimahakuto’, which has Brix 13 degree, in that ‘SWEET REI’ has higher Brix (Brix 15 to 17 degree).

‘SWEET REI’ is in fact outstanding and readily identified as being such.

It should be noted that the plant age of all the plants of ‘SWEET REI’, which were observed and photographed for botanical description, was 3 years (except for the 7-year-old mother tree of ‘SWEET REI’ in FIG. 21).

### BRIEF DESCRIPTION OF THE DRAWINGS

This new white nectarine variety is illustrated by the accompanying digital color photographs, depicting defining characteristics of the plant by the best possible color photography. Colors of foliage, flowers, fruits and other plant parts may vary from year to year depending on, among other factors, nectarine cultivation practices, light conditions, air temperature, soil fertility, etc.

FIG. 1 illustrates a whole fruit of ‘SWEET REI’ fruiting on a tree (photographed date: Sep. 1, 2017; photographed location: Tsukidate-cho, Date-city, Fukushima-prefecture, Japan; and the age of photographed ‘SWEET REI’: 3 years).

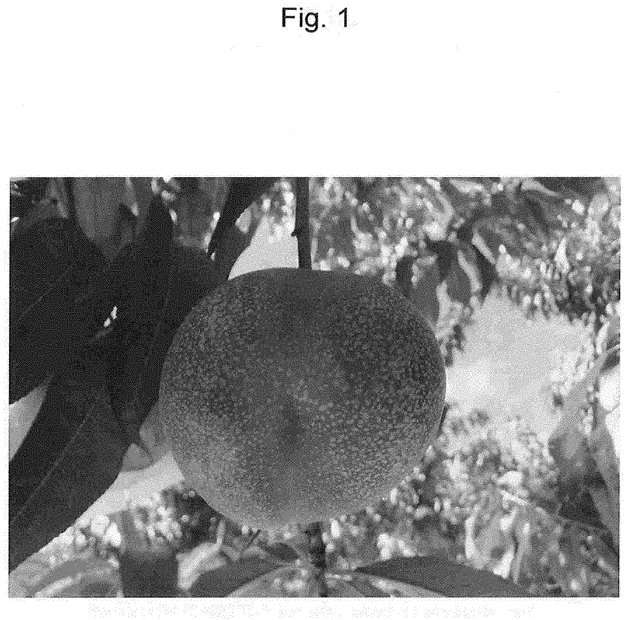

FIG. 2 illustrates apexes of 6 fruits of ‘SWEET REI’ (photographed date: Aug. 31, 2017; photographed location: Higashine-city, Yamagata-prefecture, Japan; and the age of photographed ‘SWEET REI’: 3 years).

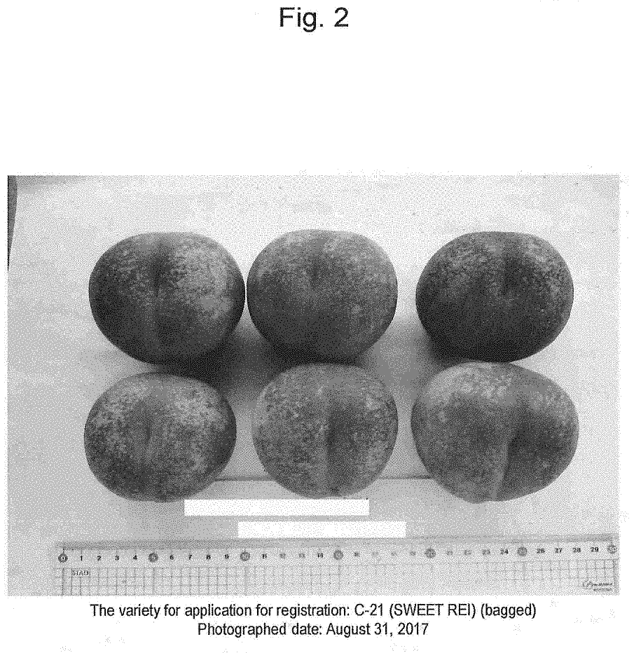

FIG. 3 illustrates lateral view of 6 fruits of ‘SWEET REI’ (photographed date: Aug. 31, 2017; photographed location: Higashine-city, Yamagata-prefecture, Japan; and the age of photographed ‘SWEET REI’: 3 years).

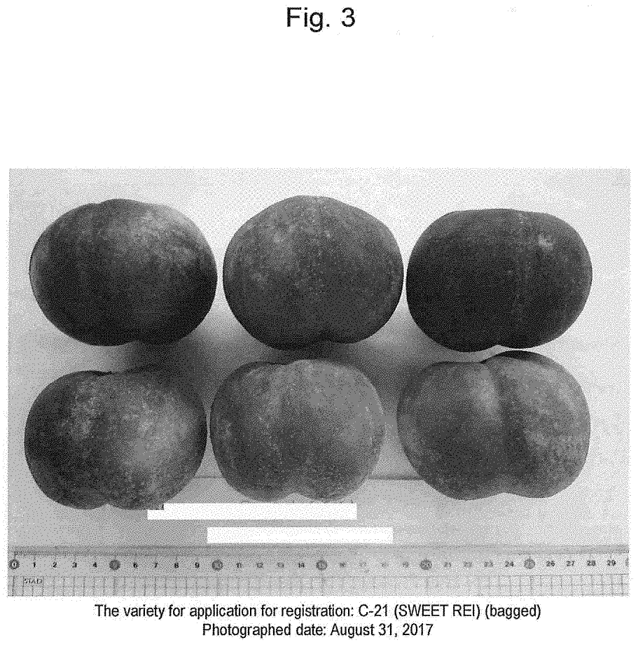

FIG. 4 illustrates stalk cavity of 6 fruits of ‘SWEET REI’ (photographed date: Aug. 31, 2017; photographed location: Higashine-city, Yamagata-prefecture, Japan; and the age of photographed ‘SWEET REI’: 3 years).

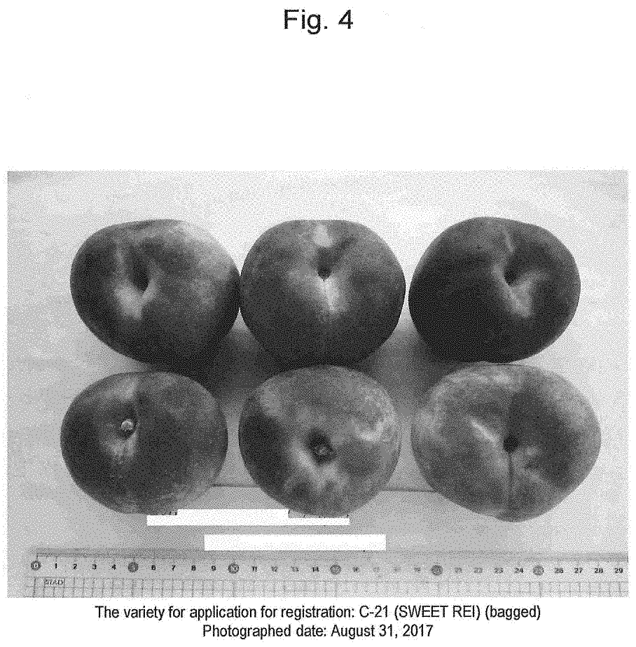

FIG. 5 illustrates 4 fruits of ‘SWEET REI’; 2 bagged fruits (left) and 2 not bagged fruits (right) (photographed date: Sep. 1, 2017; photographed location: Higashine-city, Yamagata-prefecture, Japan; and the age of photographed ‘SWEET REI’: 3 years).

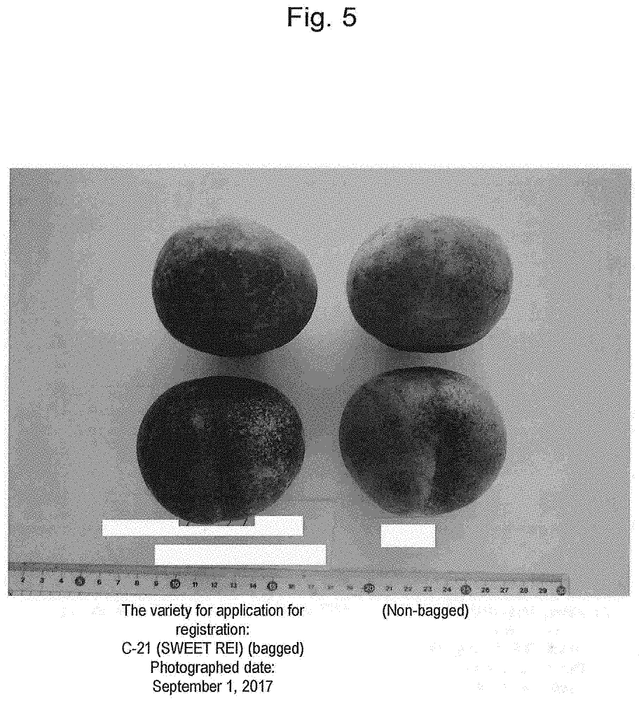

FIG. 6 illustrates 4 fruits of ‘SWEET REI’ (left), ‘SWEET VENUS’ (middle) and ‘Kawanakajimahakuto’ (right), respectively (photographed date: Sep. 1, 2017; photographed location: Higashine-city, Yamagata-prefecture, Japan; and the age of photographed ‘SWEET REI’, ‘SWEET VENUS’ and ‘Kawanakajimahakuto’: 3 years).

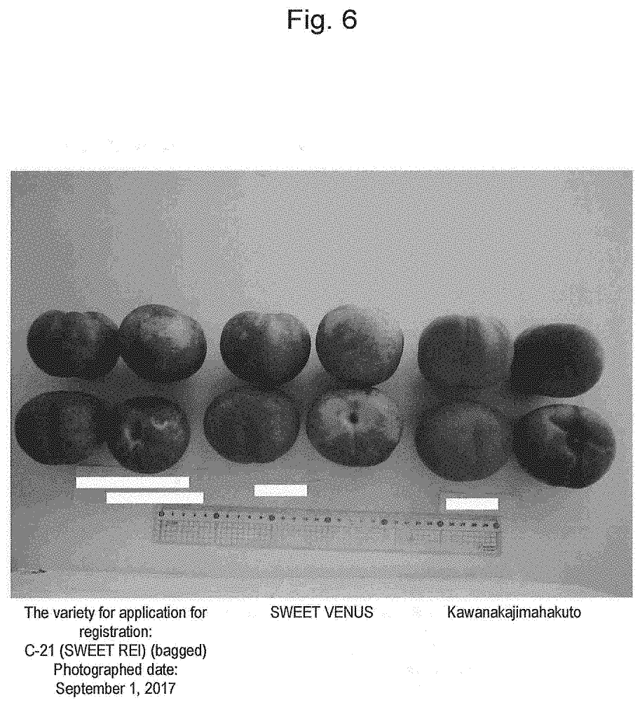

FIG. 7 illustrates longitudinal section view of fruits of ‘SWEET REI’ (photographed date: Aug. 31, 2017; photographed location: Higashine-city, Yamagata-prefecture, Japan; and the age of photographed ‘SWEET REI’: 3 years).

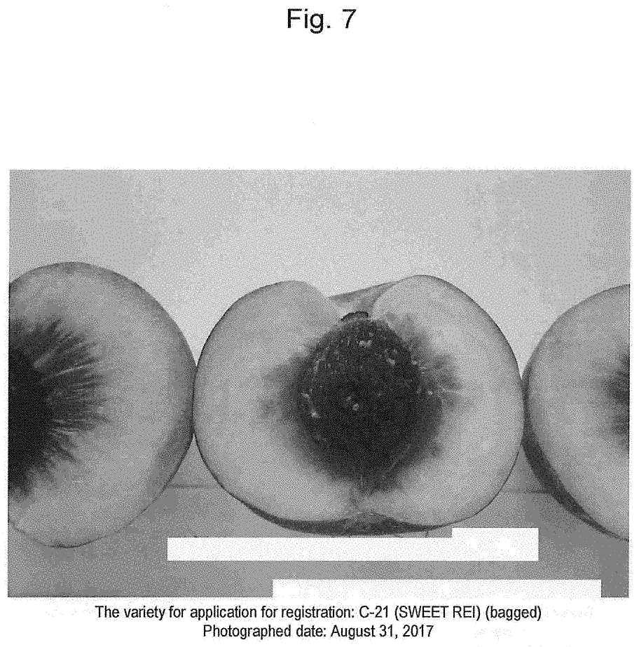

FIG. 8 illustrates cross section view of 4 fruits of ‘SWEET REI’ (left), ‘SWEET VENUS’ (middle) and ‘Kawanakajimahakuto’ (right), respectively (photographed date: Sep. 1, 2017; photographed location: Higashine-city, Yamagata-prefecture, Japan; and the age of photographed ‘SWEET REI’, ‘SWEET VENUS’ and ‘Kawanakajimahakuto’: 3 years).

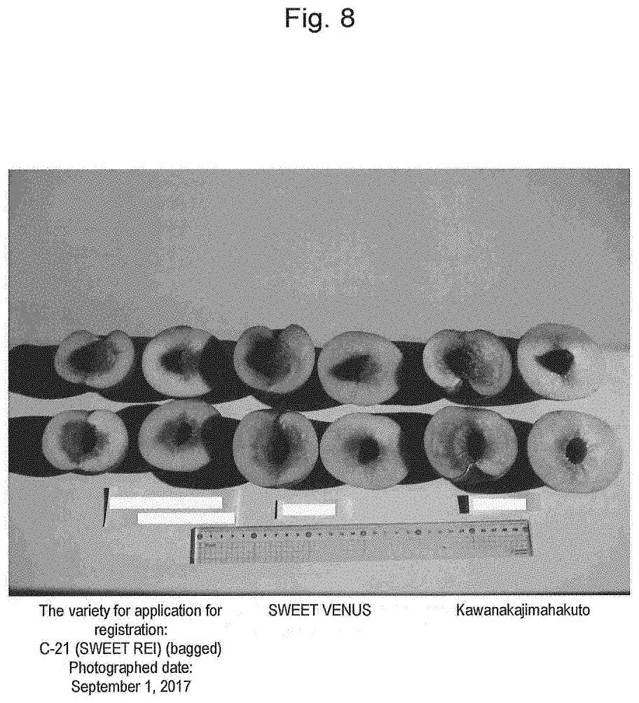

FIG. 9 illustrates 10 flowers of ‘SWEET REI’ (photographed date: Apr. 10, 2018; photographed location: Higashine-city, Yamagata-prefecture, Japan; and the age of photographed ‘SWEET REI’: 3 years).

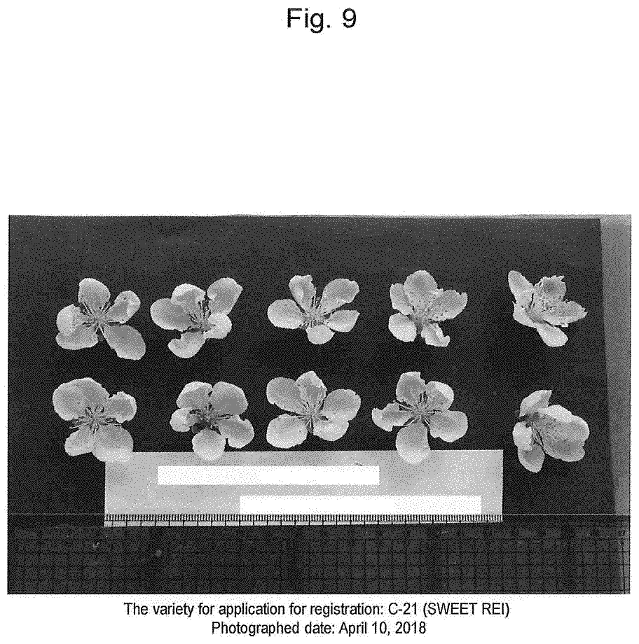

FIG. 10 illustrates 10 flowers of ‘SWEET REI’ (5 flowers before fertilization (upper) and 5 flowers with fertilized ovule (lower)) (photographed date: Apr. 10, 2018; photographed location: Higashine-city, Yamagata-prefecture, Japan; and the age of photographed ‘SWEET REI’: 3 years).

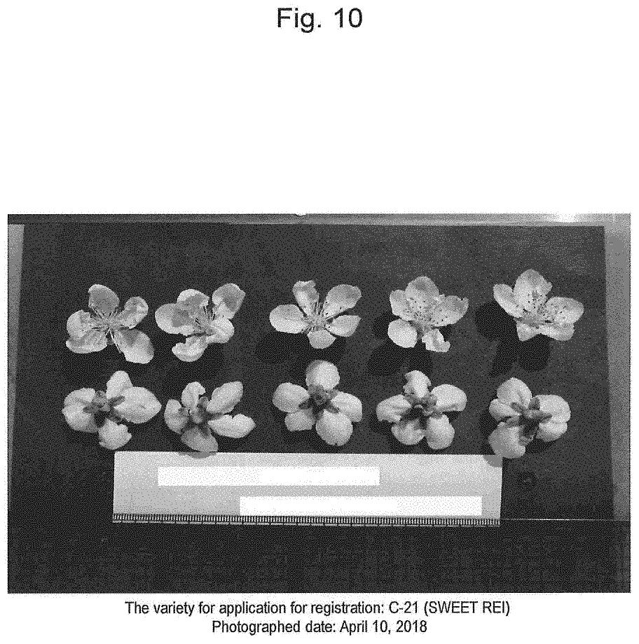

FIG. 11 illustrates 4 flowers of ‘SWEET REI’ (left), ‘SWEET VENUS’ (middle) and ‘Kawanakajimahakuto’ (right), respectively (2 flowers before fertilization (upper) and 2 flowers with fertilized ovule (lower), respectively) (photographed date: Apr. 10, 2018; photographed location: Higashine-city, Yamagata-prefecture, Japan; and the age of photographed ‘SWEET REI’, ‘SWEET VENUS’ and ‘Kawanakajimahakuto’: 3 years).

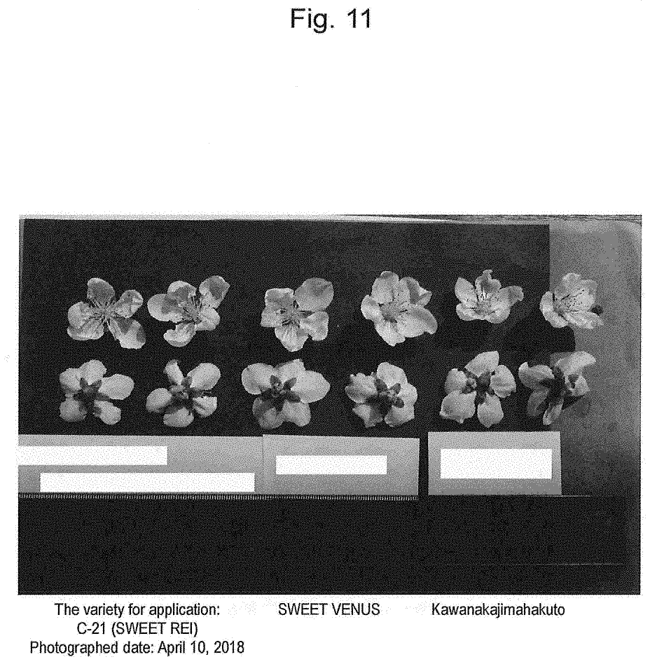

FIG. 12 illustrates a close-up longitudinal section view of a flower of ‘SWEET REI’ (photographed date: Apr. 10, 2018; photographed location: Higashine-city, Yamagata-prefecture, Japan; and the age of photographed ‘SWEET REI’: 3 years).

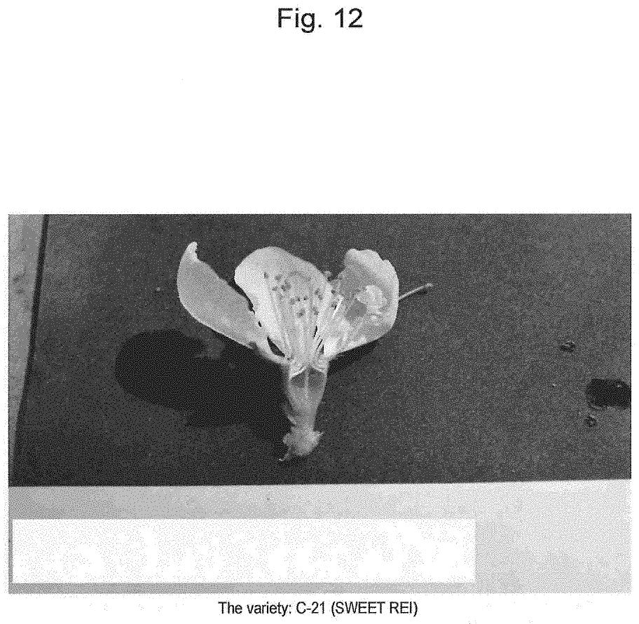

FIG. 13 illustrates a close-up longitudinal section view of a flower of ‘SWEET VENUS’ (photographed date: Apr. 10, 2018; photographed location: Higashine-city, Yamagata-prefecture, Japan; and the age of photographed ‘SWEET REI’: 3 years).

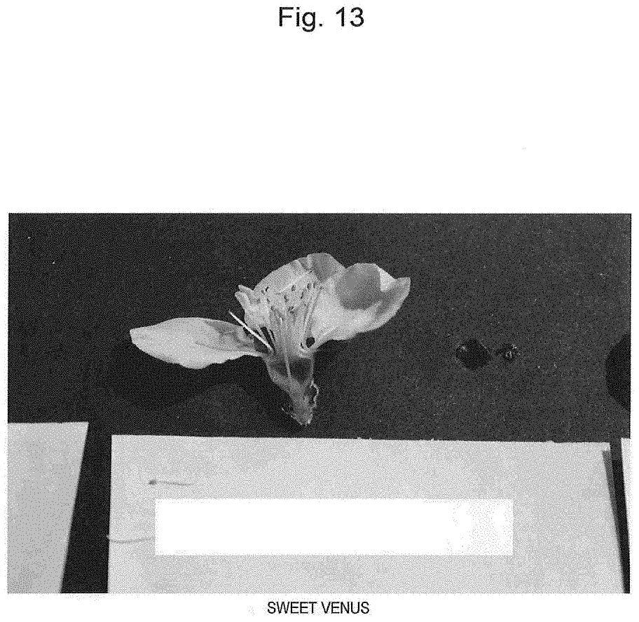

FIG. 14 illustrates a close-up longitudinal section view of a flower of ‘Kawanakajimahakuto’ (photographed date: Apr. 10, 2018; photographed location: Higashine-city, Yamagata-prefecture, Japan; and the age of photographed ‘SWEET REI’: 3 years).

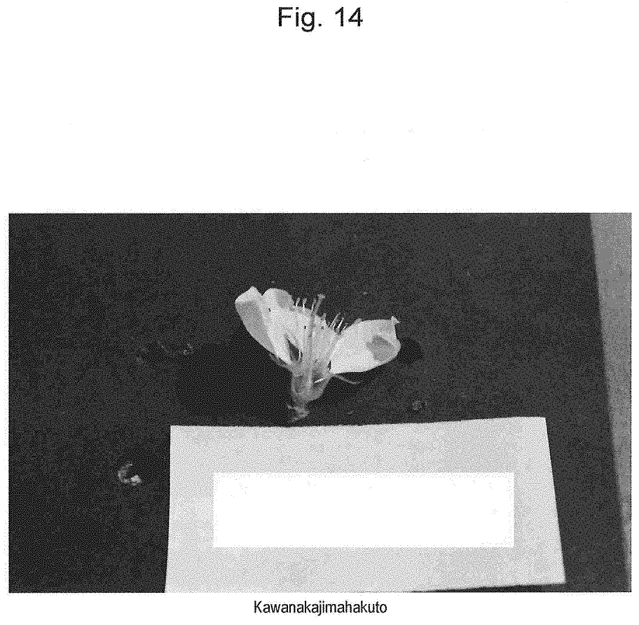

FIG. 15 illustrates 10 flower petals of ‘SWEET REI’ (upper), ‘SWEET VENUS’ (middle) and ‘Kawanakajimahakuto’ (bottom), respectively (photographed date: Apr. 10, 2018; photographed location: Higashine-city, Yamagata-prefecture, Japan; and the age of photographed ‘SWEET REI’, ‘SWEET VENUS’ and ‘Kawanakajimahakuto’: 3 years).

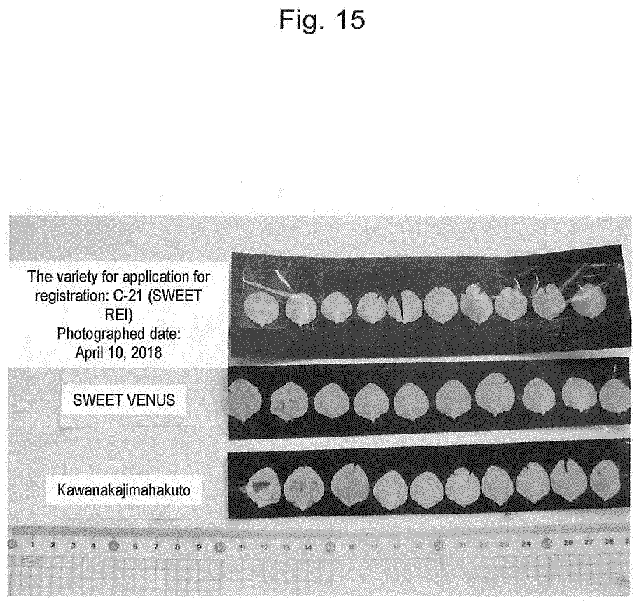

FIG. 16 illustrates a branch of ‘SWEET REI’ with a number of leaves (photographed date: Sep. 1, 2017; photographed location: Higashine-city, Yamagata-prefecture, Japan; and the age of photographed ‘SWEET REI’: 3 years).

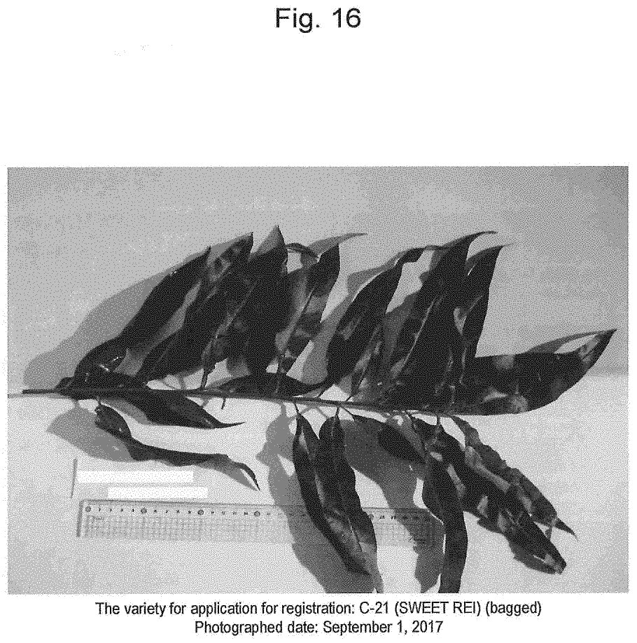

FIG. 17 illustrates a branch of ‘SWEET VENUS’ with a number of leaves (photographed date: Sep. 1, 2017; photographed location: Higashine-city, Yamagata-prefecture, Japan; and the age of photographed ‘SWEET VENUS’: 3 years).

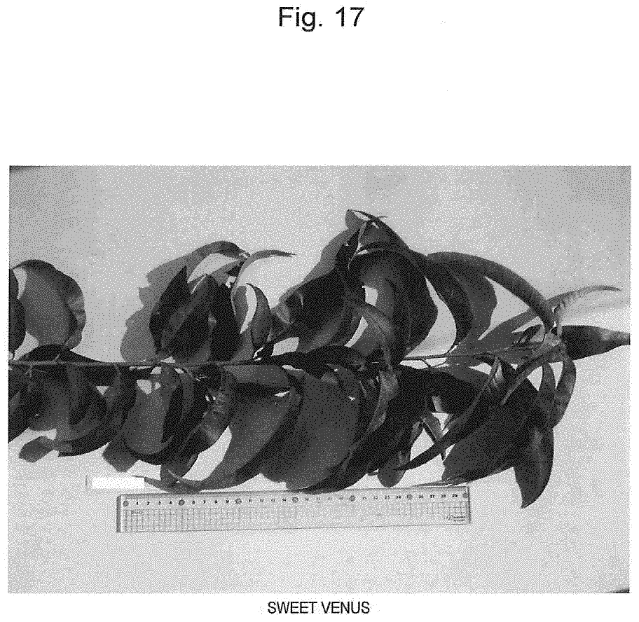

FIG. 18 illustrates a branch of ‘Kawanakajimahakuto’ with a number of leaves (photographed date: Sep. 1, 2017; photographed location: Higashine-city, Yamagata-prefecture, Japan; and the age of photographed ‘Kawanakajimahakuto’: 3 years).

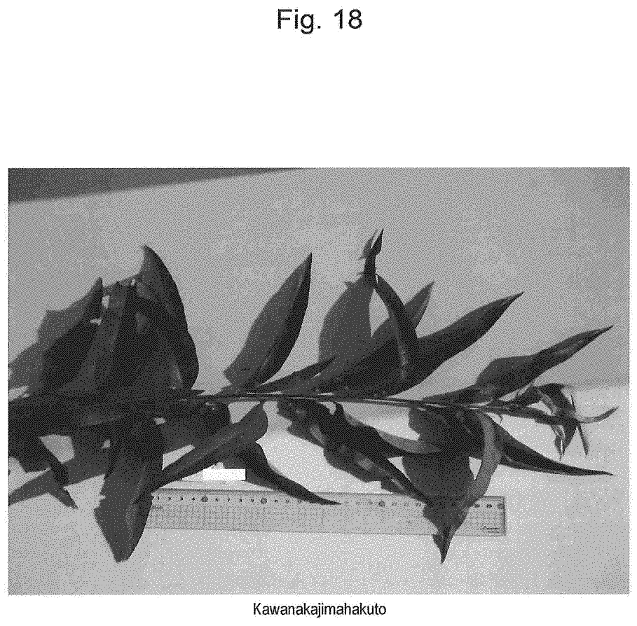

FIG. 19 illustrates 10 leaves of ‘SWEET REI’ (5 upper surface of leaves (upper) and 5 lower surface of leaves (lower)) (photographed date: Sep. 1, 2017; photographed location: Higashine-city, Yamagata-prefecture, Japan; and the age of photographed ‘SWEET REI’: 3 years).

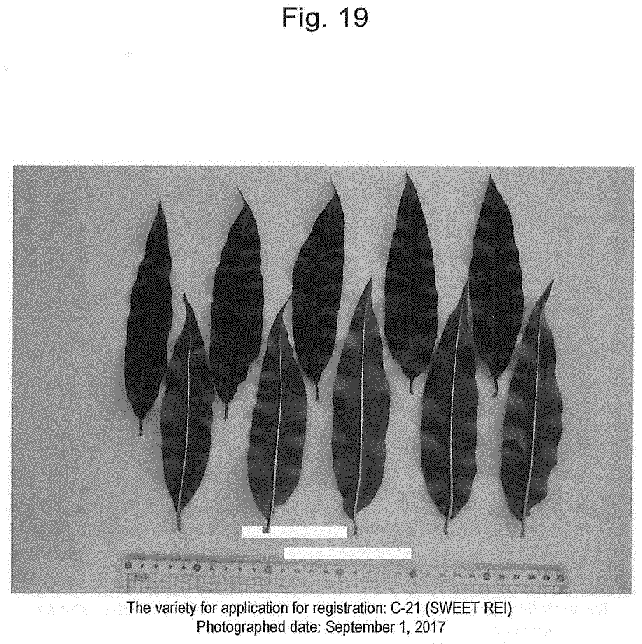

FIG. 20 illustrates 4 leaves of ‘SWEET REI’ (left), ‘SWEET VENUS’ (middle) and ‘Kawanakajimahakuto’ (right), respectively (2 upper surface of leaves (left), and 2 lower surface of leaves (right), respectively) (photographed date: Sep. 1, 2017; photographed location: Higashine-city, Yamagata-prefecture, Japan; and the age of photographed ‘SWEET REI’, ‘SWEET VENUS’ and ‘Kawanakajimahakuto’: 3 years).

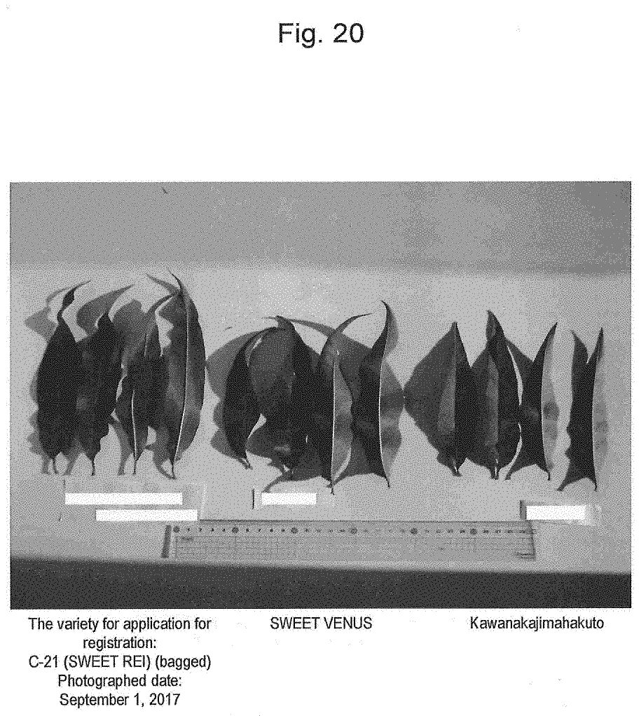

FIG. 21 illustrates a whole mother tree of ‘SWEET REI’ (photographed date: Sep. 1, 2017; photographed location: Tsukidate-cho, Date-city, Fukushima-prefecture, Japan; and the age of photographed ‘SWEET REI’: 7 years).

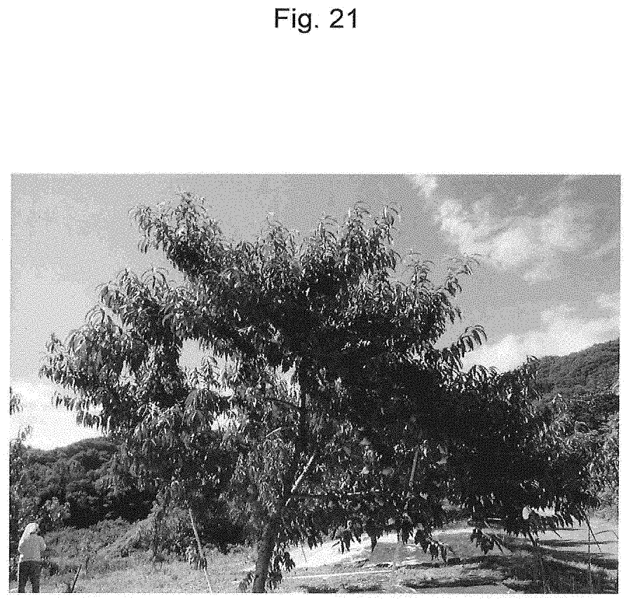

### BOTANICAL DESCRIPTION OF THE VARIETY

The following is a detailed botanical description of the new variety of white nectarine, its tree, flower, foliage and fruit, as based on observations of 3 year old specimens grown in Tsukidate-cho, Date-city, Fukushima-prefecture, Japan, with color in accordance with The R.H.S. Colour Chart 2015.
1. Tree:
   Size.—Medium (3 year old tree: Height: 2.5 to 3 m; Width: 3.5 to 4 m).
   Vigor.—Medium vigorous.
   Growth habit.—Semi-upright.
2. Shoot:
   Anthocyanin coloration of the first-year flowering shoot (excluding brindille), side away from sun.—Absent.
   Intensity of anthocyanin coloration of the first-year flowering shoot (excluding brindilles), side away from sun.—Weak.
3. Leaves:
   Time of leaf bud burst.—Medium, around March 25 (3 year old tree).
   Leaf blade length.—Average length 18.61 cm.
   Leaf blade width.—Average width 3.71 cm.
   Leaf blade ratio of length/width.—Large (average 5.02).
   Leaf blade shape in cross section.—Flat.
   Recurvature of leaf blade apex.—Absent.
   Shape (angle) at base of leaf blade.—Acute.
   Shape (angle) at apex of leaf blade.—Medium.
   Color of upper surface of mature leaf blade.—Dark Green (RHS Code: 136A).
   Color of lower surface of mature leaf blade.—Strong Yellow Green (RHS Code: 144A).
   Red mid-vein on lower surface of mature leaf blade.—Absent.
   Petiole length.—Average length 1.34 cm.
   Nectaries on petiole of leaf.—Present.
   Shape of nectaries on petiole of leaf.—Rounded and reniform.
   Predominant number of nectaries on petiole of leaf.—More than two.
4. Flower buds:
   Density of flower buds on the first-year flowering shoot.—Dense.
   General distribution of flower buds on the first-year flowering shoot.—Distributed in groups of two or more.
5. Flowers:
   Flower type.—Showy.
   Calyx color of inner side (opened flower, before falling of petals).—Strong Yellowish Green (RHS Code: N144A).
   Corolla: predominant color (inner side).—Light Purplish pink (RHS Code: 65B).
   Petal shape.—Rounded shaped.
   Petal size.—Average size 3.27 cm².
   Petal number per flower.—Five.
   Time of beginning of flowering.—Medium, around April 12 (3 year old tree).
   Duration of flowering.—Medium around April 12 to around April 24 (3 year old tree).
6. Reproductive organs:
   Stamens position compared to petals.—Below.
   Sigma position compared to anthers.—Above.
   Pollen in anthers.—Present.
   Pubescence on ovary.—Absent.
7. Fruit:
   Fruit size (weight).—Medium, Average weight: 208.7 g.
   Fruit shape in ventral view.—Oblate.
   Fruit shape of pistil end.—Weakly depressed.
   Fruit symmetry viewed from pistil end.—Asymmetric.
   Fruit: prominence of suture.—Medium.
   Fruit: depth of stalk cavity.—Medium, Average depth: 1.3 cm.
   Fruit: width of stalk cavity.—Medium.
   Fruit: ground color of fruit skin.—Light Yellow Green (RHS Code: 154D).
   Presence or absence of over color of fruit skin.—Present.
   Fruit: hue of over color of fruit skin.—Dark Red (RHS Code: 59A).
   Fruit: pattern of over color of fruit skin.—Mottled.
   Fruit: extent of over color of fruit skin.—Medium.
   Pubescence on fruit.—Absent.
   Fruit: density of pubescence.—Very sparse.
   Fruit: thickness of skin.—Thin.
   Fruit: adherence of skin to flesh.—Strong.
   Fruit: firmness of flesh.—Medium.
   Fruit: ground color of fruit flesh.—Yellowish White (RHS Code: 155B).
   Fruit: anthocyanin coloration directly under fruit skin.—Absent or very weakly expressed.
   Fruit: anthocyanin coloration of fruit flesh.—Absent or very weakly expressed.
   Fruit: anthocyanin coloration around stone.—Strongly expressed.
   Fruit: texture of fruit flesh.—Not fibrous.
   Fruit sweetness.—High, Brix 15 to 17 degree.
   Fruit acidity.—Medium.
   Time of maturity for consumption.—Late, around late August.
   Tendency to preharvest drop.—Weak.
8. Stone:
   Stone: size compared to fruit (ratio weight of stone/weight of fruit).—Small.
   Stone shape in lateral view.—Obovate.
   Stone: intensity of brown color.—Dark.
   Stone: relief of surface.—Pits and grooves.
   Stone: tendency of splitting (at peak harvest)).—Medium.
   Stone: presence or absence of adherence to fruit flesh.—Absent.
   Stone: degree of adherence to flesh.—Weak.
9. Others:
   Use.—The fruit of ‘SWEET REI’ is suitable for fruit gift, such as fruits gift box or fruits basket, since it has attractive dark-red fruit skin color as well as excellent eating quality, and is easy to eat because of its freestone flesh. In addition, due to its high brix and relatively low acidity, fruits of ‘SWEET REI’ can also be used for preparation of processed fruits, such as fruit cake, fruit jelly, canned fruits, fruit juice and fruit jam.
   Keeping quality.—Remarkable. Fruit of ‘SWEET REI’ have a slow maturation and a long shelf life both on the tree after growth completion (before harvesting) and after harvesting without alteration. After harvest, fruits are well preserved around 2 weeks, at around 4.0 degree Celsius.
   Shipping quality.—Considered very good. The fruits of ‘SWEET REI’ showed minimal bruising of the flesh or skin damage after being subjected to normal harvesting and packing procedures. Its resistance to handling during harvest and packing and its long shelf life without alteration after harvest easily permit around 2 weeks-shipping, at around 4.0 degrees Celsius.

VII. Table of Characteristics (Table of Characteristics)

| | | | | | | | ↓Sweet Rei | | ↓Sweet venue | ↓Kawanakajimahakuto | |
|---|---|---|---|---|---|---|---|---|---|---|---|
| | | | | | | | Sweet Rei | | Sweet venue | (Kawanakajimahatatto) | |
| | | | Characteristics(Characteristics) | | | | | State (State) | | | |
| Character No | UPOV No. | Symbol | (Japanese) | (English) | Definition | Method | Grade | (Japanese) | (English) | Example Variety (Ex. Var.) | Remarks |
| 1 | 1(*) | QNG | Tree: size | Tree: size | Size of whole tree | Observation | 1 | very small | very small | Silver Prolific | |
| | | | | | | | 3 | small | small | Mizuno nectarine | |
| | | | | | | | 5 | medium | medium | Kawanakajimahakuto, | 5 |
| | | | | | | | | | 6 | Mizuno nectarine | |
| | | | | | | | 7 | large | large | Akatsuki, Fantasia | |
| | | | | | | | 9 | very large | very large | Mochizuki | |
| 2 | 2 | QN | Tree: vigor | Tree: vigor | Vigor of growth of | Observation | 3 | weak | weak | Kawanakajimahakuto | 3 |
| | | | | | branches | | 5 | medium | medium | Hakuho, | |
| | | | | | | | | | 6 | Mizuno nectarine | |
| | | | | | | | 7 | strong | strong | Mochizuki, Nectared2 | |
| 3 | 3(*) | QN | Tree: habit | Tree: habit | Shape of whole tree, | Observation | 1 | upright | upright | Myojyo, Maygrand | |
| | | | | | viewed from lateral | | 3 | semi-upright | semi-upright | Akatsuki, Shuhou, | |
| | | | | | side | | | | | Hakuho | |
| | | | | | | | 5 | spreading | spreading | Ookubo, | 5 |
| | | | | | | | | | | Kawanakajimahakuto | |
| | | | | | | | 7 | drooping | drooping | | |
| | | | | | | | 9 | weeping | weeping | Terute Suimitsu | |
| 4 | 4 | QN | Flowering | Flowering shoot: | Thickness of center | Measurement | 3 | thin | thin | | |
| | | | shoot: | thickness | part of first-year | mm | 5 | medium | medium | Ookubo, Hakuto, | |
| | | | thickness | (excluding | grown shoot | | | | | Hakuho | |
| | | | | brindlilles) | | | 7 | thick | thick | | |
| 5 | 5 | QN | Flowering | Flowering shoot: | Length of intemodes | Measurement | 1 | very short | very short | Silver Prolific | |
| | | | shoot: length | length of | or center part of | cm | 3 | short | short | | |
| | | | of intemodes | intemodes | first-year grown | | 5 | medium | medium | Akatsuki, Okitsu, | |
| | | | | (excluding | shoot | | | | | Hakuho | |
| | | | | brindilles) | | | 7 | long | long | Maygrand | |
| | | | | | | | 9 | very long | very long | Mochizuki | |
| 6 | 6(*) | QL | Flowering | Flowering shoot: | Presence or absence | Observation | 1 | absent | absent | | 1 |
| | | | shoot: | anthocyanin | of anthocyanin | | 9 | present | present | | |
| | | | anthocyanin | coloration | expression in | | | | | | |
| | | | coloration | (excluding | first-year flowering | | | | | | |
| | | | | brindilles, side | shoot, side away | | | | | | |
| | | | | away from sun) | from sun | | | | | | |
| 7 | 7(*) | QN | Flowering | Flowering shoot: | Intensity of | Observation | 3 | weak | weak | | 3 |
| | | | shoot: | intensity of | anthocyanin | | 5 | medium | medium | | |
| | | | intensity of | anthocyanin | expression in a | | 7 | strong | strong | | |
| | | | anthocyanin | coloration | first-year flowering | | | | | | |
| | | | coloration | (excluding | shoot | | | | | | |
| | | | | brindilles, side | | | | | | | |
| | | | | away from sun) | | | | | | | |
| 8 | 8(*) | QN | Flowering | Flowering shoot: | density of flower | Measurement | 3 | sparse | sparse | Feicheng peach, | |
| | | | shoot: | density of | buds on a first-year | | | | | Yukimomo | |
| | | | density of | flower buds | flowering shoot | | 5 | medium | medium | Fantasia | |
| | | | flower buds | | | | 7 | dense | dense | Akatsuki, Ookubo, | 7 |
| | | | | | | | | | | Hakuto, Hakuho | |
| 9 | 9 | QL | Flowering | Flowering shoot: | General distribution | Observation | 1 | isolated | isolated | | |
| | | | shoot: | general | of flower buds on a | | 2 | in groups of | in groups of | | 2 |
| | | | general | distribution of | first-year flowering | | | two or more | two or more | | |
| | | | distribution of | flowwer buds | shoot | | | | | | |
| | | | flowwer buds | (excluding | | | | | | | |
| | | | | brindilles, side | | | | | | | |
| | | | | away from sun) | | | | | | | |
| 10 | 10(*) | QLG | Flower: type | Flower: type | Flower type in | Observation | 1 | showy | showy | Hakuto, Shuhou, | 1 |
| | | | | | flowering time | | | | | Fantasia, Hakuho | |
| | | | | | | | 2 | non showy | non showy | Myojyo, Nectared1 | |
| 11 | 11(*) | PQ | Calyx: color | Calyx: color of | Color of inner side of | Observation | 1 | greenish | greenish | Hakuho, Sotta nectarine | 1 |
| | | | of inner side | inner side | calyx-tube in | | | yellow | yellow | | |
| | | | | (opened flower, | flowering time | | 2 | orange | orange | Ougontou, Nishiki, | |
| | | | | before falling of | | | | | | Fantasia, Redheaven | |
| | | | | petals) | | | | | | | |
| 12 | 12(*) | PQ | Corolla: | Corolla: | Color of inner side of | Observation | 1 | white | white | Zansetsushidare, | |
| | | | predominant | predominant | petal in flowering | | | | | Juseito (white), | |
| | | | color | color (inner side) | time | | | | | Nakate White | |
| | | | | | | | 2 | very light pink | very light pink | | |
| | | | | | | | 3 | light pink | light pink | Nakatsuhakuto, | |
| | | | | | | | | | | Mizuno nectarine, | |
| | | | | | | | | | | Hakuho | |
| | | | | | | | 4 | medium pink | medium pink | Hakuto, Nectared4 | |
| | | | | | | | 5 | dark pink | dark pink | Sunago-wase, Fantasia | |
| | | | | | | | 6 | violet pink | violet pink | | |
| | | | | | | | 7 | red | red | Myojyo, Nectared1 | |
| 13 | 13(*) | PQ | Petal: shape | Petal: shape | Shape of petal in | Observation | 1 | narrow elliptic | narrow elliptic | Akatsuki, Tenshin, | |
| | | | | | flowering time | | | | | Nectared5 | |
| | | | | | | | 2 | broad elliptic | broad elliptic | Kawanakajimahakuto, | 2 |
| | | | | | | | | | | Yuzora, Okitsu | |
| | | | | | | | 3 | round | round | Nishiki, Ohatsumomo | |
| 14 | 14(*) | QN | Petal: size | Petal: size | Size of petal in | Measurement | 1 | very small | very small | Redheaven | |
| | | | | | flowering time | cm² | 3 | small | small | Tenshin | |
| | | | | | | | 5 | medium | medium | Hakuho, Akatsuki, | 5 |
| | | | | | | | | 3.27 cm² | 3.68 cm² | Mochizuki | 3.5 cm² |
| | | | | | | | 7 | large | large | Ookubo, Akabanabanto | |
| | | | | | | | 9 | very large | very large | | |
| 15 | 15(*) | QL | Petals: | Petals: number | Number of petals per | Measurement | 1 | 5 | five | Akatsuki, Fantasia | 1 |
| | | | number | | flower in flowering | | 2 | 6 or more | more than five | Nakate White, | |
| | | | | | time | | | | | Terute-beni | |
| 16 | 16 | QN | Stamens: | Stamens: | Relative postion of | Observation | 1 | below | below | | 1 |
| | | | position | position | stamers to petals in | | 2 | same level | same level | | |
| | | | compared to | compared to | flowering time | | 3 | above | above | | |
| | | | petals | petals | | | | | | | |
| 17 | 17(*) | QL | Stigma: | Stigma: | Relative postion of | Observation | 1 | below | below | Redheaven | |
| | | | position | position | stigma to anthers in | | 2 | same level | same level | Hakuho, Chiyohime | |
| | | | compared to | compared to | flowering time | | 3 | above | above | Tenshin | 3 |
| | | | anthers | anthers | | | | | | | |
| 18 | 18(*) | QL | Anthers: | Anthers: pollen | Presence or absence | Observation | 1 | absent | absent | Kawanakajimahakuto, | 1 |
| | | | pollen | | of pollen in flowering | | | | | Akizora | |
| | | | | | time | | 9 | present | present | Hakuho, | |
| | | | | | | | | | | Hikawahakuho, Akatsuki | |
| 19 | 19(*) | QL | Ovary: | Ovary: | Presence or absence | Observation | 1 | absent | absent | | |
| | | | pubescence | pubescence | of pubescence on | | 9 | present | present | | 9 |
| | | | | | ovary in flowering | | | | | | |
| | | | | | time | | | | | | |
| 20 | 20 | QN | Young shoot: | Young shoot: | Length of stipule on | Measurement | 3 | short | short | | |
| | | | length of | length of stipule | center part of | mm | 5 | medium | medium | | |
| | | | stipule | | first-year shoot | | 7 | long | long | | |
| 21 | 21(*) | QN | Leaf blade: | Leaf blade: | Longitudinal length | Measurement | 3 | short | short | Kawanakajimahakuto, | 3 |
| | | | length | length | of a mature leaf on | cm | | | | Mochizuki | 13.2 cm |
| | | | | | center part of | | 5 | medium | medium | Hakuto, Ookubo, Okitsu | |
| | | | | | first-year shoot | | | | 16.61 cm | | |
| | | | | | (excluding petiole) | | 7 | long | long | Akatsuki, Flavor Top | |
| | | | | | | | | 18.61 cm | | | |
| 22 | 22(*) | QN | Leaf blade: | Leaf blade: | Maximum width of | Measurement | 3 | narrow | narrow | Mochizuki, | 4 |
| | | | width | width | mature leaf on center | cm | | 3.71 cm | 3.94 cm | Harrow Blood | 3.35 cm |
| | | | | | part of first-year | | 5 | medium | medium | Hakuho | |
| | | | | | shoot | | 7 | broad | broad | Akizora | |
| 23 | 23(*) | QN | Leaf blade: | Leaf blade: | Ratio of | Measurement | 3 | small | small | Ohatsumomo, | 4 |
| | | | ratio length/ | ratio length/ | length/maximum | | | | | Okinawa, Akashidare | 3.95 cm |
| | | | width | width | width of mature leaf | | 5 | medium | medium | Ookubo, Redheaven, | |
| | | | | | on center part of | | | 5.02 cm | 4.22 cm | Hakuho | |
| | | | | | first-year shoot | | 7 | large | large | Silver Prolific | |
| 24 | 24 | PQ | Leaf blade: | Leaf blade: | Shape in | Observation | 1 | concave | concave | | 1 |
| | | | shape in cross | shape in cross | cross-section of | | 2 | flat | flat | | |
| | | | section | section | center part of mature | | 3 | convex | convex | | |
| | | | | | leaf on center part of | | | | | | |
| | | | | | first-year shoot | | | | | | |
| 25 | 25 | QL | Leaf blade: | Leaf blade: | Recurvature of leaf | Observation | 1 | absent | absent | Hakuho, Akatsuki | 1 |
| | | | recurvature of | recurvature of | blade apex of mature | | 9 | Present | Present | Redheaven | |
| | | | apex | apex | leaf on center part of | | | | | | |
| | | | | | first-year shoot | | | | | | |
| 26 | 26 | PQ | Leaf blade: | Leaf blade: | Shape of mature leaf | Observation | 1 | acute | acute | Golden Proflific | |
| | | | angle at base | angle at base | base on center part | | 2 | approximately | approximately | Hakuho, Hakuto, | |
| | | | | | of first-year shoot | | | right angle | right angle | Early rawford | |
| | | | | | | | 3 | obtuse | obtuse | Elberta, Redheaven, | 3 |
| | | | | | | | | | | Shanghai, Shimizu | |
| 27 | 27 | QN | Leaf blade: | Leaf blade: | Size of angle at apex | Observation | 3 | small | small | Golden Proflific, | |
| | | | angle at apex | angle at apex | or mature leaf on | | | | | kikumomo, Houkimomo | |
| | | | | | center part of | | 5 | medium | medium | Akatsuki, Hakuto, | 5 |
| | | | | | first-year shoot | | | | | Ookubo, Elberta | |
| | | | | | | | 7 | large | large | Salway, Tuskan, | |
| | | | | | | | | | | Independence, | |
| | | | | | | | | | | Akashidare, | |
| | | | | | | | | | | Sagamishidare | |
| 28 | 28 | PQ | Leaf blade: | Leaf blade: | Color of mature leaf | Observation | 1 | greenish | greenish | Ougentou, Fantasia | |
| | | | color | color | on center part of | | | yellow | yellow | | |
| | | | | | first-year shoot | | 2 | green | green | New Yorker, Hakuho | 2 |
| | | | | | | | 3 | purplish red | purplish red | Akame, Peach | |
| | | | | | | | | | | Rootstock Tsukuba 4 | |
| 29 | | QL | Leaf: red | Leaf: red | Red mid-vein on the | Observation | 1 | absent | absent | | |
| | | | mid-vein on | mid-vein on | lower side of mature | | 9 | Present | Present | | |
| | | | the lower side | the lower side | leaf on center part of | | | | | | |
| | | | | | first-year shoot | | | | | | |
| 30 | 29 | QN | Petiole: | Petiole: | Length of petiole of | Measurement | 3 | short | short | Elberta, Fantasia, | 3 |
| | | | length | length | mature leaf on center | mm | | | | Akashidare | 0.6 cm |
| | | | | | part of first-year | | 5 | medium | medium | Hakuho, Akatsuki, | |
| | | | | | shoot | | | 1.34 cm | 1.09 cm | Nectared7, Nectared8, | |
| | | | | | | | | | | Kanmomo No. 5 | |
| | | | | | | | 7 | long | long | Swatow, Golden Glory, | |
| | | | | | | | | | | Juseito (red) | |
| 31 | 30(*) | QLG | Petiole: | Petiole: | Presence or absence | Observation | 1 | Absent | Absent | | |
| | | | nectaries | nectaries | of nectaries on | | 9 | present | present | | 9 |
| | | | | | petiole immediately | | | | | | |
| | | | | | after completion of | | | | | | |
| | | | | | extension of petiole | | | | | | |
| 32 | 31(*) | PQ(+) | Petiole: | Petiole: | Shape of Nectaries | Observation | 1 | round | round | Saotome, Sungrand | |
| | | | shape of | shape of | on petiole | | 2 | reniform | reniform | Ougontou, Fantasia | 2 |
| | | | nectaries | nectaries | | | | | | | |
| 33 | 32 | QL | Petiole: | Petiole: | Predominant number | Measurement | 1 | 2 | two | Kawanakajimahakuto, | 1 |
| | | | predominant | predominant | of nectaries on | | | | | Ougontou | |
| | | | number of | number of | petiole | | 2 | 3 or more | more than two | Mochizuki, Nishiki, | |
| | | | nectaries | nectaries | | | | | | Ookubo | |
| 34 | 33(*) | QN | Fruit: size | Fruit: size | Weight of mature | Measurement | 1 | very small | very small | Ohatsumomo | |
| | | | | | fruit | g | 3 | small | small | Saotome | |
| | | | | | | | 5 | medium | medium | Hikawahakuho, Gyosei | |
| | | | | | | | | 208.7 g | | | |
| | | | | | | | 7 | large | large | Kawanakajimahakuto, | 7 |
| | | | | | | | | | 247.4 g | Ookubo | 235.7 g |
| | | | | | | | 9 | very large | very large | Abehakuto | |
| 35 | 34(*) | PQ(+) | Fruit: shape | Fruit: shape (in | Shape of mature | Observation | 1 | broad oblate | broad oblate | Ooberibanto | |
| | | | | ventral view) | fruit, viewed from | | 2 | oblate | oblate | Gyosei, Nishiki, | 2 |
| | | | | | front side (suture | | | | | Nectared2 | |
| | | | | | side) | | 3 | round | round | Ookubo, | |
| | | | | | | | | | | Hiratsuka Red, Hakuho | |
| | | | | | | | 4 | ovate | ovate | Hikawahakuho, Fantasia | 4 |
| | | | | | | | 5 | eliptic | eliptic | Early Sun Grand, | |
| | | | | | | | | | | Tenshin | |
| 36(*) | 35 | PQ | Fruit: shape | Fruit: shape | Shape of fruit apex | Observation | 1 | prominently | prominently | Tenshin, Harrow Blood | |
| | | | of pistil end | of pistil end | of mature fruit | | | pointed | pointed | | |
| | | | | | | | 2 | weakly pointed | weakly pointed | Sweet Gold | |
| | | | | | | | 3 | flat | flat | Chiyomaru, | |
| | | | | | | | | | | Kushigatahakuto | |
| | | | | | | | 4 | weakly | weakly | Saotome, Nishiki, | |
| | | | | | | | | depressed | depressed | Mochizuki | |
| | | | | | | | 5 | strongly | strongly | Hikawahakuho | |
| | | | | | | | | depressed | depressed | | |
| 37 | 36 | QL(+) | Fruit: | Fruit: symmetry | Symmetry of mature | Observation | 1 | asymmetric | asymmetric | Ookubo, Aichihakuto | |
| | | | synmetry | (viewed from | fruit, viewed from | | 2 | symmetric | symmetric | Kawanakajimahakuto | 2 |
| | | | | pistil end) | fruit apex | | | | | | |
| 38 | 37 | QN | Fruit: | Fruit: | Depth of suture at | Observation | 3 | weak | weak | Masahime, Fantasia | 4 |
| | | | prominence of | prominence of | equalorial part of | | 5 | medium | medium | Akatsuki, Hakuho, | |
| | | | suture | suture | mature fruit | | | | | Okitsu | |
| | | | | | | | 7 | strong | strong | Tenshin | |
| 39 | 38 | QN(+) | Fruit: depth of | Fruit: depth of | Depth of stalk cavity | Measurement | 3 | shallow | shallow | Hakuho, Nishiki, | |
| | | | stalk cavity | stalk cavity | of mature fruit | cm | | | | Nunomewase, | |
| | | | | | | | | | | Nectared2 | |
| | | | | | | | 5 | medium | medium | Sunago-wase, Okitsu | 5 |
| | | | | | | | | 1.3 cm | 1.3 cm | | 1.5 cm |
| | | | | | | | 7 | deep | deep | Hakuto, Fantasia, | |
| | | | | | | | | | | Mochizuki | |
| 40 | 39 | QN | Fruit: width of | Fruit: width of | Width of stalk cavity | Measurement | 3 | narrow | narrow | Tenshin, Flame Kist | |
| | | | stalk cavity | stalk cavity | of mature fruit | cm | 5 | medium | medium | Akatsuki, Fantasia, | 5 |
| | | | | | | | | | | Hakuho | |
| | | | | | | | 7 | broad | broad | Mochizuki, Flavor Top, | |
| | | | | | | | | | | Kanmomo No. 5 | |
| 41 | 40(*) | PQ | Fruit: ground | Fruit: ground | Ground color of fruit | Observation | 1 | green | green | | |
| | | | color | color | skin of mature fruit | | 2 | cream green | cream green | | |
| | | | | | | | 3 | greenish white | greenish white | New Yorker, Robin | 3 |
| | | | | | | | 4 | cream white | cream white | Hakuho, Hakuto | |
| | | | | | | | 5 | cream | cream | | |
| | | | | | | | 6 | pink white | pink white | | |
| | | | | | | | 7 | greenish | greenish | | |
| | | | | | | | | yellow | yellow | | |
| | | | | | | | 8 | cream yellow | cream yellow | | |
| | | | | | | | 9 | yellow | yellow | Nakayama kinto, | |
| | | | | | | | | | | Fantasia | |
| | | | | | | | 10 | orange yellow | orange yellow | Nishiki, Oukihi | |
| 42 | 41 | QL | Fruit: over | Fruit: over | Presence or absence | Observation | 1 | absent | absent | Mochizuki | |
| | | | color | color | of over color of fruit | | 9 | present | present | | 9 |
| | | | | | skin of mature fruit | | | | | | |
| 43 | 42 | PQ | Fruit: hue of | Fruit: hue of | Hue of over color of | Observation | 1 | orange red | orange red | | |
| | | | over color | over color | fruit skin of mature | | 2 | pink | pink | Hakushu, Shimizuhakuto | |
| | | | | | fruit | | 3 | pink red | pink red | | |
| | | | | | | | 4 | light red | light red | Silver Profific | |
| | | | | | | | 5 | medium red | medium red | Akatsuki, | |
| | | | | | | | | | | Mizuno nectarine | |
| | | | | | | | 6 | dark red | dark red | Kawanakajimahakuto, | 6 |
| | | | | | | | | | | Fantasia | |
| 44 | 43(*) | PQ | Fruit: pattern | Fruit: pattern | Pattern of over color | Observation | 1 | solid flesh | solid flesh | Fantasia | |
| | | | of over color | of over color | of fruit skin of mature | | 2 | striped | striped | Akatsuki, Silver Star | |
| | | | | | fruit | | 3 | mottled | mottled | Hakuto, Silver Prolific, | 3 |
| | | | | | | | | | | Hakuho | |
| | | | | | | | 4 | marbled | marbled | Chiyomaru, | |
| | | | | | | | | | | Takeihakuho, Ookubo | |
| 45 | 44(*) | QN | Fruit: extent | Fruit: extent | Extent of over color | Observation | 1 | absent or very | absent or very | Mochizuki | |
| | | | of over color | of over color | on mature fruit | | | small | small | | |
| | | | | | | | 3 | small | small | Hakuto, Silver Prolific, | |
| | | | | | | | 5 | medium | medium | Sotta nectarine | |
| | | | | | | | 7 | large | large | Akatsuki, Hiratsuka Red | 7 |
| | | | | | | | 9 | very large | very large | Kawanakajimahakuto, | |
| | | | | | | | | | | Fantasia | |
| 46 | 45(*) | QLG | Fruit: | Fruit: | Presence or absence | Observation | 1 | absent | absent | Shuhou, Flavor Top | |
| | | | pubescence | pubescence | of pubecence on | | 9 | present | present | | 9 |
| | | | | | surface of mature | | | | | | |
| | | | | | fruit | | | | | | |
| 47 | 46(*) | QN | Fruit: density | Fruit: density | Density of | Observation | 1 | very sparse | very sparse | | |
| | | | or pubescence | or pubescence | puescence on | | 3 | sparse | sparse | Saotome | |
| | | | | | surface of mature | | 5 | medium | medium | Hakuho, Hakuto, | 5 |
| | | | | | fruit | | | | | Kawanakajimahakuto, | |
| | | | | | | | 7 | dense | dense | Nishiki | |
| | | | | | | | 9 | very dense | very dense | | |
| 48 | 47 | QN | Fruit: | Fruit: | Thickness of fruit | Measurement | 3 | thin | thin | Hakuho, Ookubo, | |
| | | | thickness of | thickness of | skin of mature fruit | | | | | Hakuto | |
| | | | skin | skin | | | 5 | medium | medium | | 5 |
| | | | | | | | 7 | thick | thick | | |
| 49 | 48 | QN | Fruit: | Fruit: | Adherence of fruit | Observation | 1 | absent or very | absent or very | | |
| | | | adherence of | adherence of | skin to fruit flesh | | | weak | weak | | |
| | | | skin to flesh | skin to flesh | | | 3 | weak | weak | Nunomewase, | |
| | | | | | | | | | | Kushigatahakuto, | |
| | | | | | | | | | | Hakuho | |
| | | | | | | | 5 | medium | medium | Akatsuki, | 5 |
| | | | | | | | | | | Kawanakajimahakuto, | |
| | | | | | | | 7 | strong | strong | Sweet Gold, | |
| | | | | | | | | | | Kanmomo No. 5 | |
| | | | | | | | 9 | very strong | very strong | Manami, Odoroki | |
| 50 | 49(*) | QN | Fruit: | Fruit: | Firmness of fruit | Measurement | 1 | very soft | very soft | Nunomewase, | |
| | | | firmness of | firmness of | flesh at equatorial | | 3 | soft | soft | Hakuho, Chiyomaru | |
| | | | flesh | flesh | part of mature fruit | | 5 | medium | medium | Akatsuki, | 5 |
| | | | | | | | | | | Kawanakajimahakuto, | |
| | | | | | | | 7 | firm | firm | Nishiki, Kanmomo No. 5 | |
| | | | | | | | 9 | very firm | very firm | Odoroki, Manami | |
| 51 | 50(*) | PQC | Fruit: ground | Fruit: ground | ground color of fruit | Observation | 1 | greenish white | greenish white | Nagano-yaseito (Late) | |
| | | | color of flesh | color of flesh | flesh of mature fruit | | 2 | white | white | Sotta nectarine | |
| | | | | | | | 3 | cream white | cream white | Hakuto, Yamatohakuto, | 3 |
| | | | | | | | | | | Hakuho | |
| | | | | | | | 4 | light yellow | light yellow | Mochizuki | |
| | | | | | | | 5 | yellow | yellow | Ougontou, Maygrand | |
| | | | | | | | 6 | orange yello | orange yello | Kanmomo No. 12 | |
| | | | | | | | 7 | orange | orange | | |
| | | | | | | | 8 | red | red | | |
| 52 | 51(*) | QN | Fruit: | Fruit: | Intensity of | Observation | 1 | absent or very | absent or very | Mochizuki | 1 |
| | | | anthocyanin | anthocyanin | anthocyanin | | | weakly | weakly | | |
| | | | coloration | coloration | coloration at | | | expressed | expressed | | |
| | | | directly under | directly under | immediately under | | 2 | weakly | weakly | Shimizuhakuto | |
| | | | skin | skin | fruit skin of mature | | | expressed | expressed | | |
| | | | | | fruit | | 3 | strongly | strongly | Yoshihime, | |
| | | | | | | | | expressed | expressed | Kawanakajimahakuto, | |
| | | | | | | | | | | Tenshin, Harrow Blood | |
| 53 | 52(*) | QN | Fruit: | Fruit: | Intensity of | Observation | 1 | absent or very | absent or very | Mochizuki, Maygrand | |
| | | | anthocyanin | anthocyanin | anthocyanin | | | weakly | weakly | | |
| | | | coloration of | coloration of | coloration in fruit | | | expressed | expressed | | |
| | | | flesh | flesh | flesh of mature fruit | | 2 | weakly | weakly | Shimizuhakuto | 2 |
| | | | | | | | | expressed | expressed | Hiratsuka Red | |
| | | | | | | | 3 | strongly | strongly | Yoshihime, Okitsu, | |
| | | | | | | | | expressed | expressed | Tenshin, Harrow Blood | |
| 54 | 53(*) | QN | Fruit: | Fruit: | Intensity of | Observation | 1 | absent or very | absent or very | Mochizuki | |
| | | | anthocyanin | anthocyanin | anthocyanin | | | weakly | weakly | | |
| | | | coloration | coloration | coloration around | | | expressed | expressed | | |
| | | | around stone | around stone | stone of mature fruit | | 2 | weakly | weakly | Shimizuhakuto, Nishiki, | |
| | | | | | | | | expressed | expressed | Okitsu | |
| | | | | | | | 3 | strongly | strongly | Hakuho, Akatsuki, | 3 |
| | | | | | | | | expressed | expressed | Kawanakajimahakuto, | |
| 55 | 54 | QL | Fruit: texture | Fruit: texture | Texture of fruit flesh | Observation | 1 | not fibrous | not fibrous | Shimizuhakuto, | 1 |
| | | | of the flesh | of the flesh | atequatorial part of | | | | | Hiratsuka Red, Hakuto | |
| | | | | | mature fruit | | 2 | fibrous | fibrous | Ookubo, New Yorker | |
| 56 | 55 | QN | Fruit: | Fruit: | Degree of sweetness | Measurement | 3 | low | low | Silver Prolific | |
| | | | sweetness | sweetness | of fruit flesh at | | 5 | medium | medium | Chiyohime, Nectared2, | 5 |
| | | | | | equatorial part of | | | | | Hakuho | |
| | | | | | mature fruit | | 7 | high | high | Akatsuki, | |
| | | | | | | | | | | Kawanakajimahakuto, | |
| | | | | | | | | | | Okitsu | |
| 57 | 56 | QN | Fruit: acidity | Fruit: acidity | Degree of acidity of | Measurement | 3 | low | low | Hakuho, | 3 |
| | | | | | fruit flesh at | | | | | Kawanakajimahakuto, | |
| | | | | | equatorial part of | | 5 | medium | medium | Ougontou, Shuhou | |
| | | | | | mature fruit | | 7 | high | high | tenshin, Fantasia, | |
| | | | | | | | | | | Elberta, Redhaeaven | |
| 58 | 57(*) | QN | Stone: size | Stone: size | Relative wight of | Measurement | 3 | small | small | Saotome, Nishiki | |
| | | | compared to | compared to | stone to mature fruit | | 5 | medium | medium | Hakuho, Akatsuki | 5 |
| | | | fruit | fruit | (weight of | | 7 | large | large | Ookubo, Flavor Top | |
| | | | | | stone/whole weight | | | | | | |
| | | | | | of fruit) | | | | | | |
| 59 | 58(*) | | Stone: shape | Stone: shape | Shape in lateral view | Observation | 1 | oblate | oblate | | |
| | | | in lateral view | (in lateral view) | (n0n-suture side) of | | 2 | round | round | Saotome, Mochizuki | |
| | | | | | stone of mature fruit | | 3 | eliptic | eliptic | Hakuho, Akatsuki, | 3 |
| | | | | | | | | | | Shuhou | |
| | | | | | | | 4 | obovate | obovate | | |
| 60 | 59 | QN | Stone: | Stone: | Intensity of brown | Observation | 3 | light | light | Chiyomaru, Maygrand | |
| | | | intensity of | intensity of | color of stone of | | 5 | medium | medium | Hakuho, Akatsuki, | |
| | | | brown color | brown color | mature fruit | | | | | Okitsu | |
| | | | | | | | 7 | dark | dark | Kawanakajimahakuto, | 7 |
| | | | | | | | | | | fantasia | |
| 61 | 60 | PQ(+) | Stone: relief | Stone: relief | Relief of surface of | Observation | 1 | small pits | small pits | | |
| | | | of surface | of surface | stone of mature fruit | | 2 | large pits | large pits | | |
| | | | | | | | 3 | grooves | grooves | Shuhou, Fantasia | |
| | | | | | | | 4 | pits and | pits and | Hakuho | 4 |
| | | | | | | | | grooves | grooves | | |
| 62 | 61 | QN | Stone: | Stone: | Tendency of splitting | Measurement | 1 | absent or | absent or | Kawanakajimahakuto, | |
| | | | tendency of | tendency of | of stone of mature | | | low | low | Mochizuki | |
| | | | splitting | splitting (at | fruit | | 3 | low | low | Akatsuki, Fantasia | |
| | | | | peak harvest) | | | 5 | medium | medium | Hikawahakuho, | 5 |
| | | | | | | | | | | Maygrand | |
| | | | | | | | 7 | high | high | Sunago-wase, | |
| | | | | | | | | | | Nectared1 | |
| | | | | | | | 9 | very high | very high | | |
| 63 | 62(*) | QLG | Stone: | Stone: | Presence or absence | Observation | 1 | absent | absent | | |
| | | | adherence to | adherence to | of adherence of | | 9 | present | present | | 9 |
| | | | flesh | flesh | stone (or free stone) | | | | | | |
| | | | | | to fruit flesh of | | | | | | |
| | | | | | mature fruit | | | | | | |
| 64 | 63 | QN | Stone: | Stone: | Degree of adherence | Observation | 3 | weak | weak | Ougontou, Fantasia | |
| | | | degree of | degree of | of stone (or free | | 5 | medium | medium | Nunomewase, | |
| | | | adherence to | adherence to | stone) to fruit flesh of | | | | | Maygrand | |
| | | | flesh | flesh | mature fruit | | 7 | strong | strong | Akatsuki, Hakuto, | 7 |
| | | | | | | | | | | LeGrand, Hakuho | |
| 65 | 64 | QN | Time of leaf | Time of leaf | Timing of first leaf | Observation | 1 | very early | very early | Okinawa | |
| | | | bud burst | bud burst | bud burst | | 3 | early | early | Nishiki, Flavor Top | |
| | | | | | | | 5 | medium | medium | Hakuho, Nectared | 5 |
| | | | | | | | 7 | late | late | Ookubo, Hiratsuka Red | |
| | | | | | | | 9 | very late | very late | | |
| 66 | 65(*) | QNG | Time of | Time of | Time of beginning | Observation | 1 | very early | very early | Okinawa, Coral | |
| | | | beginning of | beginning of | of sequential | | 3 | early | early | Nishiki, Shuhou, | |
| | | | flowering | flowering | flowering | | | | | Flavor Top | |
| | | | | | | | 5 | medium | medium | Akatsuki, Chiyohime | |
| | | | | | | | 7 | late | late | Hakuho, | |
| | | | | | | | | | | Mizuno nectarine | |
| | | | | | | | 9 | very late | very late | Kawanakajimahakuto, | 9 |
| 67 | 66(*) | QN | Duration of | Duration of | Length of duration of | Observation | 3 | short | short | | |
| | | | flowering | flowering | flowering | | 5 | medium | medium | | 5 |
| | | | | | | | 7 | long | long | | |
| 68 | 67(*) | QNG | Time of | Time of | Timing of maturity of | Observation | 1 | very early | very early | Hatsuotome, Fukuotome | |
| | | | maturity for | maturity for | fruit for consumption | | 3 | early | early | Sunago-wase, | |
| | | | consumption | consumption | | | | | | Hikawahakuho | |
| | | | | | | | 5 | medium | medium | Hakuho, Akatsuki, | |
| | | | | | | | | | | Ookubo | |
| | | | | | | | 7 | late | late | Kawanakajimahakuto, | 7 |
| | | | | | | | | | | Fantasia | |
| | | | | | | | 9 | very late | very late | Akizora, Nishio Gold | |
| 69 | 68 | QN | Tendency to | Tendency to | Tendency to | Observation | 1 | absent or | absent or | | |
| | | | preharvest | preharvest | preharvest | | | weak | weak | | |
| | | | drop | drop | drop | | 3 | weak | weak | Akatsuki, | 3 |
| | | | | | | | | | | Kawanakajimahakuto, | |
| | | | | | | | | | | Shuhou | |
| | | | | | | | 5 | medium | medium | Akizora, Mochizuki | |
| | | | | | | | | | | Okitsu | |
| | | | | | | | 7 | strong | strong | | |
| | | | | | | | 9 | very strong | very strong | | |

What is claimed is:

1. A new and distinct variety of white nectarine (Prunus persica (L.) Batsch var. nucipersica (Suckow) C. K. Schneid.) denominated ‘SWEET REI’, as illustrated and described herein.
